(19) United States
(12) Patent Application Publication
(10) Pub. No.: US 2024/0065591 A1
(43) Pub. Date: Feb. 29, 2024

(54) CANNULA INSERTION SYSTEM

(71) Applicant: Vitestro Holding B.V., Utrecht (NL)

(72) Inventors: Toon Olaf Overbeeke, Utrecht (NL); Brian Robert Joseph, 'S-Graven-Hage (NL); Johannes Gerhard Daniël Karssen, Utrecht (NL); Arris Cornelis Jonker, Utrecht (NL); Antonius Gerardus Leonardus Hoevenaars, Utrecht (NL)

(21) Appl. No.: 18/259,504

(22) PCT Filed: Dec. 28, 2021
(86) PCT No.: PCT/EP2021/087752

§ 371 Date: Jun. 27, 2023

(30) Foreign Application Priority Data
Dec. 29, 2020 (NL) 2027242

(51) Int. Cl. A61B 5/15; A61B 5/00

(52) U.S. Cl. CPC A61B 5/150748; A61B 5/150389; A61B 5/489

(57) ABSTRACT

The invention provides a cannula insertion system to insert a cannula into a blood vessel of a human or animal body, comprising: ⋅an ultrasound probe (3) to provide an ultrasound signal to determine a location of a blood vessel (V) in a human or animal body, ⋅a cannula insertion device (8) configured to insert the cannula (2) into the blood vessel, ⋅wherein the ultrasound probe comprises a contact surface (3b) to contact a skin (S) of the human or animal body, ⋅wherein the contact surface comprises one or more protrusions (14, 16) that can be used to stabilize the blood vessel when the contact surface is pressed on the skin.

The present invention relates to a cannula insertion system into a blood vessel of a human or animal body. The cannula insertion system may for example be used to semi-automatically or automatically insert a cannula, for instance a needle, into the blood vessel to withdraw blood. In practice, it may be challenging to penetrate the wall of the blood vessel, because the wall of the blood vessel is cylindrical and made of relatively hard tissue compared with surrounding tissue of the human or animal body. Moreover, the blood vessel may laterally shift with respect to the skin when the cannula is inserted into the human or animal body, for example when the cannula is pushed on the relatively hard wall of the blood vessel. This lateral shifting is also referred to as rolling of the blood vessel.

US2017/035335 discloses a device to maintain a user's blood vessel in position during insertion of a needle into the blood vessel. The device includes two branches separated by a gap of width greater than the size of the blood vessel. A positioner is provided to position the branches around the blood vessel. The positioner may comprise an arm configured to move in translation along three axes a rotation device for rotating the branches. The device can additionally include a heater to heat at least one branch and a controller to control the heater. The device can further include a cooler to cool at least one branch and the controller is configured to activate the cooler.

The positioner of the device is needed to position the branches opposite sides of the blood vessel in the appropriate positions to stabilize a blood vessel in the human or animal body. The positioner requires additional equipment. Further, the maneuvering of the branches to the desired location requires space that should be made available within the space surrounding the insertion location. This space occupied by the positioner of the branches cannot be used for other components of the cannula insertion system. As a result, the design of the cannula insertion system may be more challenging.

The present invention provides a cannula insertion system as claimed in claim 1.

The cannula insertion system comprises an ultrasound probe that can measure an ultrasound signal that can be used to obtain ultrasound images. To obtain a suitable ultrasound signal a contact surface of the ultrasound probe should be pressed on the skin of the human or animal body. By providing one or more protrusions on the contact surface of the ultrasound probe, the contact surface can be used to stabilize the blood vessel when the contact surface is pressed on the skin, the penetration of the needle can be more accurately performed with respect to the blood vessel, since the blood vessel is stabilized in its position.

The one or more protrusions can be arranged next to at least one side of the blood vessel and pressed on the skin of the human or animal body to define a surface that prevents a lateral shift of the blood vessel in at least one lateral direction, e.g. in a radial direction of the cross section of the blood vessel substantially parallel to the skin surface of the human or animal body.

The advantage of the cannula insertion system of the invention is that no separate branches, as disclosed in US2017/035335, and a positioner to position the branches are required to obtain effective stabilization of a respective blood vessel. This has the additional advantage that the position of the branches does not have to be separately controlled and no space is needed for maneuvering the branches to a desired location.

Another advantage of the one or more protrusion on the ultrasound probe is that the ultrasound probe may be generally pressed with more force on the skin of the human or animal body without compressing the blood vessel. This is in particular useful since the imaging quality of the ultrasound probe may substantially improve with increasing pressure with which the ultrasound probe is pressed on the skin. At the same time, increasing this pressure on the blood vessel will compress the cross section of the blood vessel which will make the placement of the end of the cannula in the blood vessel more difficult. In particular in the case of a superficial blood vessel, i.e. a blood vessel located closely to the skin, the compression of the blood vessel by the ultrasound probe may substantially complicate the insertion of the cannula in the blood vessel. In the cannula insertion system of the present invention, the superficial blood vessel may be received by the recess. As a result the compression of the blood vessel is substantially reduced.

In an embodiment, the one or more protrusions comprise a first protrusion and a second protrusion, wherein the first protrusion and the second protrusion are spaced with respect to each other to provide a recess therebetween in which a blood vessel can be stabilized. The first and second protrusion define a recess in which the blood vessel can be stabilized. In this way the blood vessel is held between two side surfaces preventing lateral shift in two opposite direction. This provides a very efficient stabilization of the blood vessel. In an alternative embodiment, only one protrusion may be provided to provide a stabilization surface at one side of the blood vessel.

In an embodiment, the ultrasound probe comprises an elongate sensor measurement area extending in a sensor measurement area direction, wherein the first protrusion and the second protrusion are arranged on a line extending parallel to the sensor measurement area direction. The contact surface of the ultrasound probe is the part of the ultrasound system that is designed to make contact, at least partially, with the skin of the human or animal body to obtain signals to construct ultrasound images of tissue below the skin. This contact surface comprises a sensor measurement area. This sensor measurement area is the actual surface of the ultrasound probe through which an ultrasound transducer obtains sensor signals that are used to construct ultrasound images. The sensor measurement area is typically an elongate area extending in a sensor measurement area direction. During ultrasound measurements, the sensor measurement area direction is typically arranged in a plane extending in a direction substantially perpendicular to the longitudinal direction of the blood vessel of interest.

By placing the first protrusion and the second protrusion on a line extending parallel to the sensor measurement area direction, the first protrusion and the second protrusion can be arranged at opposite sides of the blood vessel, while substantially maintaining this measurement position of the ultrasound probe with respect to the blood vessel.

In an embodiment, the first protrusion and the second protrusion are arranged on the sensor measurement area. By arranging the first protrusion and the second protrusion on the sensor measurement area the recess in which the blood vessel is stabilized is also arranged on the sensor measurement area. This has the advantage that the position of the blood vessel can be controlled accurately at a known location. An additional advantage is that the sensor measurement area can be pressed relatively more firmly against the skin of the human or animal body without compression of the blood vessel which is located in or below the recess formed between the first protrusion and the second protrusion.

In an embodiment, the line extending parallel to the sensor measurement area direction is spaced from the sensor measurement area. In this embodiment, the sensor measurement area may be free from the first protrusion and the second protrusion so that the measurement field of the ultrasound transducer is not obstructed by the presence of the first protrusion and the second protrusion. This position of the first protrusion and the second protrusion has the additional advantage that by rotation of the ultrasound probe around the sensor measurement area direction, a pressure difference between a first pressure with which the sensor measurement area and a second pressure with which the first protrusion and the second protrusion are pressed against the skin of the human or animal body can be adapted. The first pressure is related to the measurement quality of the ultrasound probe, while the second pressure is related to the degree of stabilization of the blood vessel but also the degree of compression of the blood vessel. By rotation of the ultrasound probe around the sensor measurement area direction this pressure difference can be controlled to optimize the first pressure and the second pressure.

In this embodiment, the first protrusion and the second protrusion are preferably arranged at a side of the sensor measurement area where the cannula insertion device is located, but may also be located at an opposite side of the sensor measurement area.

In an alternative embodiment, the first protrusion and the second protrusion may each extend from the sensor measurement area to a position spaced from the sensor measurement area. In such embodiment, the advantages of the first protrusion and the second protrusion being arranged on the sensor measurement area and being arranged on a line extending parallel to the sensor measurement area direction spaced from the sensor measurement area are combined. It is also possible to provide two sets of a first protrusion and a second protrusion, the first set being arranged on the sensor measurement area and the second set being arranged on a line extending parallel to the sensor measurement area direction spaced from the sensor measurement area to have these combined advantages. In yet another embodiment, it is possible that the first protrusion and the second protrusion each extend from a position spaced at one side of the sensor measurement area over the sensor measurement area to a position spaced at an opposite side of the sensor measurement area. Also two sets of a first protrusion and a second protrusion may be provided, wherein the first set is arranged on a line extending parallel to the sensor measurement area direction spaced at one side of the sensor measurement area and the second set is arranged on a line extending parallel to the sensor measurement area direction spaced at an opposite side of the sensor measurement area.

In an embodiment, the cannula insertion device is arranged to insert the cannula into the blood vessel at a cannula insertion location, and wherein the first protrusion and the second protrusion are positioned to be pressed on the skin at opposite sides of the cannula insertion location.

In an embodiment, the cannula insertion system further comprises:
a cannula insertion device positioning system to support and position the cannula insertion device,
an ultrasound probe positioning system to support and position the ultrasound probe, and
a control device arranged to control the cannula insertion device positioning system and the ultrasound probe positioning system such that during insertion of the cannula into the blood vessel the cannula insertion device positioning system positions the cannula insertion device to insert the cannula into the blood vessel at a cannula insertion location and the ultrasound probe positioning system positions the first protrusion and the second protrusion on the skin at opposite sides of the cannula insertion location.

In an embodiment, the ultrasound probe comprises a housing and an exchangeable probe cap mounted on the housing and forming at least part of the contact surface of the ultrasound housing. By using a probe cap on the ultrasound probe the shape of the contact surface may be adapted without the need to provide a completely adapted shape of the housing of the ultrasound probe. The probe cap may for example be held on the housing by a snap-fit connection, another form-fit connection, or by clamping of the probe cap on the housing.

In an embodiment, the one or more protrusions are integrated in the probe cap. By integrating the one or more protrusions in the probe cap, the housing of the ultrasound probe does not have to be specifically adapted for the provision of the one or more protrusions. This may for example be advantageous when it is desired to provide different configurations of the one or more protrusions. By exchange of the probe cap the number, location, shape and/or size of the one or more protrusions can be easily changed, for example in relation to the specific needs of a specific application. The optimal configuration of the one or more protrusions may for example be dependent on the size of an animal or person, e.g. a child or an adult, and other factors such as a fat percentage. By providing probe caps with different configurations, the ultrasound probe can easily be adapted to the specific animal or human body by selecting the probe cap with the most suitable configuration of the one or more protrusions.

In an embodiment, the one or more protrusions are integrated in the housing and the probe cap covers the one or more protrusions. In this embodiment, the housing of the ultrasound probe is provided with the one or more protrusions. The probe cap may for example be provided as an hygienic barrier that prevents direct contact between the skin of the human or animal body and the housing of the ultrasound probe. The probe cap may be provided with additional protrusions at the same or at other locations than the one or more protrusions in the housing of the ultrasound probe.

In an embodiment, the probe cap completely forms the contact surface of the ultrasound probe. When the probe cap is used as an exchangeable hygienic barrier between the housing of the ultrasound probe and the human or animal body, advantageously no openings are provided in probe cap. In the probe cap, at least at the location aligned with the sensor measurement area, a means or device may be provided to improve the transmission of the ultrasound waves of the ultrasound probe through the probe cap. For example, a gel pad may be integrated in the probe cap at the location aligned with the sensor measurement area.

In another embodiment, the probe cap comprises an opening to partially or completely expose a sensor measurement area of the ultrasound probe. The presence of the probe cap on the sensor measurement area may have a negative effect on the measurement quality of the ultrasound probe. By providing an opening to partially or completely expose the sensor measurement area, the sensor measurement area may be directly placed on the skin of the human or animal body. At the same time, the probe cap may be used to provide one or more protrusions in the contact surface of the ultrasound probe.

The probe cap may be made of polypropylene or another material that efficiently transmits ultrasound waves.

In an alternative embodiment, the ultrasound probe is provided without a probe cap, whereby the one or more protrusions are integrated in a housing of the ultrasound probe.

In an embodiment of the ultrasound probe, the cannula insertion system may comprise one or more force sensors that are arranged to measure the force with which the one or more protrusions are pressed on the skin of the human or animal body. On the basis of this/these measured force(s) and/or the ultrasound signals obtained by the ultrasound probe a blood vessel shape may be monitored. In particular, it can be determined whether the force is not too low such that there is an increased risk on rolling of a blood vessel and/or whether the force is not too high such that there is a risk that the blood vessel is compressed too much for insertion of a cannula into the blood vessel.

Generally, it may be advantageous to apply some pressure on the blood vessel such that the cross section of the blood vessel will have an oval shape with a width which is larger than a height of the blood vessel. It has been found that a blood vessel having a cross-section with this shape will have less tendency to roll/shift laterally than a blood vessel having a round shape. The shape deformation can be controlled by determining the shape of the cross section of the blood vessel in ultrasound images obtained with the ultrasound probe and/or by measuring the force exerted by the one or more protrusions on the skin of the human or animal body, comparing the determined shape of the blood vessel with a desired shape and adjusting, when needed, the pressure applied by the ultra sound probe on the blood vessel to adapt the shape of the blood vessel to the desired shape. Any other way of determining the shape of the cross section of the blood vessel may also be applied.

In an embodiment, the cannula insertion system is arranged to control a pressure with which the ultrasound probe is pressed on the human or animal body on the basis of a value representative for a non-roundness of the blood vessel, for example a ratio between a height and width of the blood vessel.

For example, the cannula insertion system may be configured to apply a pressure on the blood vessel such that a ratio between height and width of the blood vessel is in the range 0.5 to 0.9, such as in the range of 0.6 to 0.8. Such non-round shape will have less tendency to roll. At the same time, the cross section of the blood vessel will have sufficient height to introduce the cannula into the interior of the blood vessel. Any other value representative for a non-roundness of the blood vessel may also be used to control the pressure that is applied on the blood vessel by the ultra sound probe.

The invention further provides an ultrasound probe to provide an ultrasound signal to determine a location of a blood vessel in a human or animal body, wherein the ultrasound probe comprises a contact surface to contact a skin of the human or animal body, wherein the contact surface comprises one or more protrusions that can be used to stabilize the blood vessel when the contact surface is pressed on the skin. This ultrasound probe may have optionally the features described with respect to the ultrasound probe of the cannula insertion system described in this patent application.

Further characteristics and advantages of the cannula insertion system of the invention will now be explained by description of an embodiment of the invention, whereby reference is made to the appended drawings, in which:

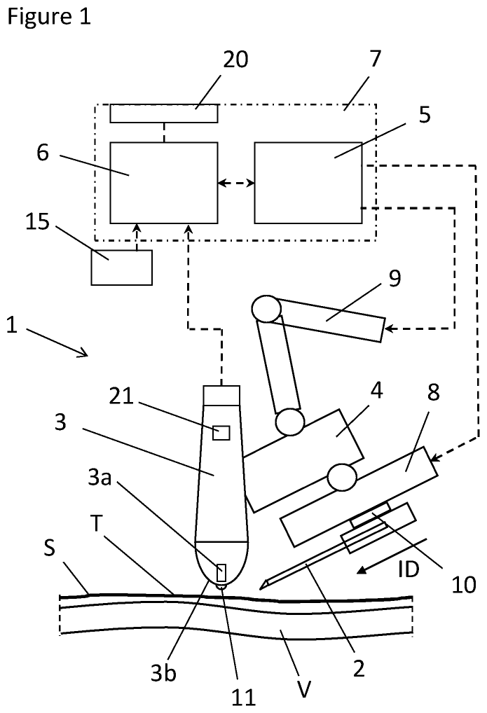

FIG. 1 shows schematically a cannula insertion system according to an embodiment of the invention comprising an end-effector supporting an ultrasound probe and a cannula insertion device;

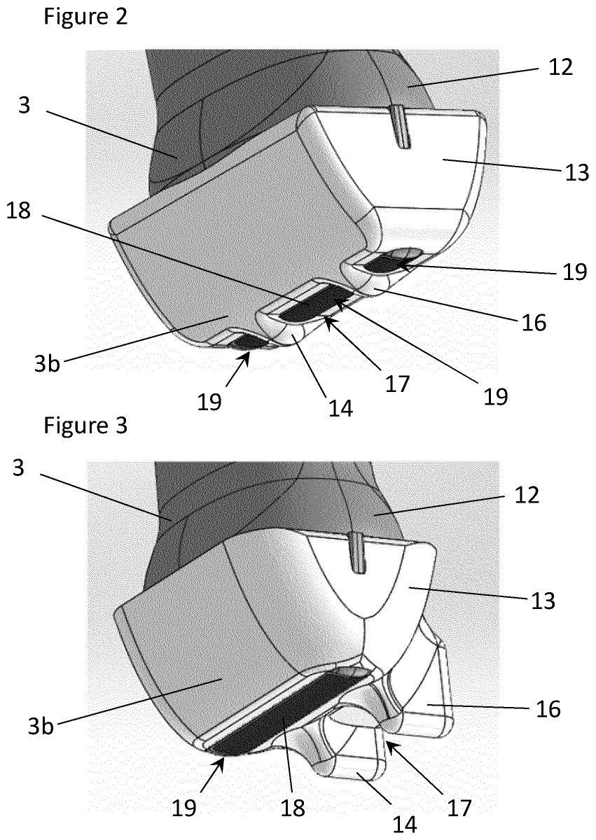

FIG. 2 shows a first embodiment of an ultrasound probe according to the invention;

FIG. 3 shows a second embodiment of an ultrasound probe according to the invention;

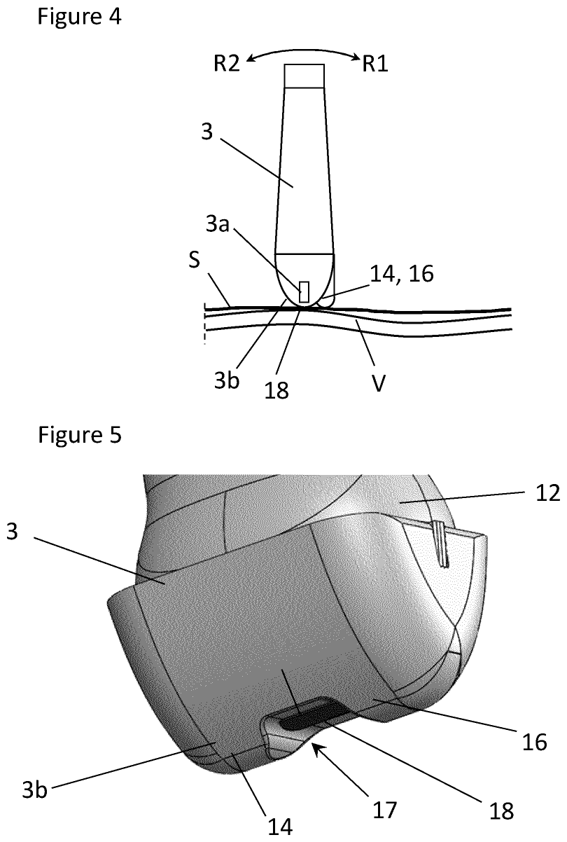

FIG. 4 shows the effect of rotation of the ultrasound probe; and

FIG. 5 shows a third embodiment of an ultrasound probe according to the invention.

FIG. 1 shows a cannula insertion system, generally denoted by reference numeral 1. The cannula insertion system 1 is configured to autonomously insert a cannula 2, for example a needle into a blood vessel V of a human or animal. The cannula insertion system 1 as shown in FIG. 1 is in particular configured to autonomously draw blood from a blood vessel V, such as a vein. In alternative embodiments, the cannula insertion system 1 may be configured to arrange a cannula 2 in a blood vessel V for intravenous medication and/or infusions.

To draw blood from the blood vessel V, the cannula insertion system 1 may be arranged to determine a location of a blood vessel V underneath the skin S of the human or animal, insert a cannula 2 into the blood vessel V and draw blood from the blood vessel V without direct interaction of an operator of the cannula insertion system 1.

The cannula insertion system 1, shown on FIG. 1, comprises an ultrasound probe 3 comprising an ultrasound transducer 3a to obtain one or more sensor signals that are representative for the location of a blood vessel V in the human or animal. The ultrasound transducer 3a is a contact sensor comprising a contact surface 3b that, during use, at least partially may be placed on the skin S in order to obtain relevant data with respect to the location of the blood vessel V. The contact surface 3b of the ultrasound probe 3 may be guided along the skin S of the human or animal in a target area T. This target area T is an area of the skin S underneath which the presence of a blood vessel V suitable for insertion of a cannula 2 is expected and which is examined by the ultrasound probe 3. A control device 5 controls the position of the ultrasound probe 3.

The target area T may be determined by obtaining images of the skin of a human or animal, for example using infrared, NIR (near infrared) or visible light sensors 15, and determining on the basis of the images an area in which it is likely that a blood vessel V suitable for insertion of a cannula 2 will be found. The cannula insertion system 1 has multiple fixed sensors, i.e. sensors mounted at a fixed location, such as sensor 15. These multiple fixed sensors may be used to determine the target area, i.e. an approximate location of a blood vessel V that can be used for insertion of the cannula 2. In an alternative embodiment, one or more of these infrared, NIR or visible light sensors may be arranged on a movable end-effector 4.

In addition, or as an alternative the fixed sensors 15 may be used to determine a location of the elbow pit of the arm and/or the shape of the arm. The sensors 15 could also be used to detect areas which should not be used for venipuncture, like birthmarks and wounds.

Once a target area T is determined, the ultrasound transducer 3a may be used to provide a sensor signal representative for a location of the blood vessel V within the target area T. The sensor signal is fed into a processing device 6 which is arranged to process the sensor signal. The control device 5 and the processing device 6 may be comprised in a single processer 7, such as a PC.

On the basis of the sensor signal provided by the ultrasound transducer 3a, an image of the blood vessel V may be created by the processing device 6. This image may be based on multiple 2D images along a line in a certain direction, 3D coordinates and direction, 6D coordinates or may be a 3D image.

The processing device 6 determines on the basis of the sensor signal, or sensor signals, the location of a blood vessel V suitable for the insertion of the cannula 2. On the basis of this location, the processing device 6 may determine an insertion path for insertion of the cannula 2 into the blood vessel V. The processing device 6 may also locate nerves and prevent the cannula 2 from penetrating those nerves, when selecting an insertion path for the cannula 2.

If no suitable location for insertion of the cannula 2 can be found, the cannula insertion system 1 may request the patient to place the other arm in the cannula insertion system 1.

The cannula 2 is arranged on a cannula insertion device 8. The cannula 2 is held by a cannula holder 10. The cannula 2 can be taken out of the cannula holder 10 to facilitate exchange of cannulas 2.

The cannula insertion device 8 is arranged to insert the cannula 2 in an insertion direction ID through the skin S and into the blood vessel V along the insertion path determined by the processing device 6. The cannula insertion device 8 comprises an actuator to adjust an insertion angle of the cannula 2 to align a longitudinal axis of the cannula 2 with the insertion direction ID. A linear actuator is provided to translate the cannula 2 along an insertion path in the insertion direction ID.

The insertion path of the cannula 2 may be adapted to anatomical structures in the arm of the patient. By adapting the position of the cannula insertion device 8 during insertion of the cannula 2, the cannula 2 may also be moved along a non-linear insertion path.

The cannula insertion device 8 is supported by a positioning system 9 that is arranged to bring the cannula insertion device 8 in a position from which the cannula insertion device 8 may move the cannula 2 along the insertion path. The cannula insertion device 8 and the positioning system 9 are controlled by the control device 5.

In the embodiment shown in FIG. 1, the ultrasound probe 3 and the cannula insertion device 8 are mounted on a single end-effector 4. This ensures a fixed spatial relationship between the ultrasound probe 3 and the cannula insertion device 8. The positioning system 9 is therefore used for positioning of both the ultrasound probe 3 and the cannula insertion device 8. In an alternative embodiment, the ultrasound probe 3 may be held in a fixed spatial relationship with the cannula 2, such that the tip of the cannula 2 can be tracked very accurately.

Similarly, the cannula insertion system 1 may comprise an operator interface, such as a display device or communication device to provide relevant information with respect to the cannula insertion procedure to the operator. This operator interface may for example provide a sound or visual alarm system to inform the operator that the patient requires help. This operator interface may comprise a digital communication device that sends data to a remote device, e.g. a smart watch or tablet device.

The cannula insertion system 1 comprises a patient or treatment identification device 20. The patient or treatment identification device 20 is configured to read an identification of the patient. The patient or treatment identification device 20 may comprise any suitable reader or scanner to read/scan the information provided by the identification of the user.

In some cases, a blood vessel V positioned below the ultrasound probe 3 may laterally displace due to the pressure exerted with the contact surface 3b of the ultrasound probe 3 and/or the force exerted by the cannula 2 on the wall of the blood vessel V. This may also be referred to as a ‘rolling vein’. This lateral displacement is undesirable as this movement will make insertion of a cannula 2 into the blood vessel V more difficult and sometimes even impossible.

To prevent the lateral displacement of the blood vessel V wherein the contact surface 3b comprises one or more protrusions 11 that can be used to stabilize the blood vessel V when the contact surface 3b is pressed on the skin.

FIG. 2 shows a first embodiment of an ultrasound probe 3 comprising a housing 12 and a probe cap 13 which is arranged with a snap-fit connection on the housing 12. The probe cap 13 forms a substantial part of the contact surface 3b of the ultrasound probe 3. On the probe cap a first protrusion 14 and a second protrusion 16 are provided, wherein the first protrusion 14 and the second protrusion 16 are spaced with respect to each other to provide a recess 17 therebetween. When the contact surface 3b is arranged on the skin S of a human or animal body a blood vessel V below the skin can be stabilized by placing the first protrusion 14 and the second protrusion 16 on opposite sides of the blood vessel V, such that the blood vessel is arranged in or below the recess 17. This will stabilize the position of the blood vessel, i.e. will reduce the risk that the blood vessel will shift laterally due to the pressure that is exerted on the blood vessel V by the contact surface 3b of the ultrasound probe 3 and/or the cannula 2 that is pressed on the wall of the blood vessel for penetration of the wall.

The first protrusion 14 and the second protrusion 16 are integrated in the probe cap 13. This means that the size, shape, location and number of protrusions may be changed by replacement of the probe cap 13 without the need to provide a completely different probe design.

The ultrasound probe 3 comprises an elongate sensor measurement area 18 extending in a sensor measurement area direction. This sensor measurement area 18 is the area of the ultrasound probe 3, where the ultrasound transducer 3a may obtain ultrasound signals that can be used to construct ultrasound images of the human or animal body on which the contact surface 3b is placed.

In the embodiment of FIG. 2, the first protrusion 14 and the second protrusion 16 are arranged on the sensor measurement area 18. This has the advantage that the blood vessel V is stabilized at the location where the ultrasound measurements are made.

Advantageously, the cannula insertion location, i.e. the location where the cannula insertion device 8 will insert the cannula 2 into the blood vessel V is also selected to be between the first protrusion 14 and the second protrusion 16.

The probe cap 13 comprises three openings 19 to expose the sensor measurement area 18 of the ultrasound probe 3. Only at the locations where the first protrusion 14 and the second protrusion 16 are provided the sensor measurement area 18 is covered by the probe cap 13.

In an alternative embodiment, the probe cap 13 may completely define the contact surface 3b of the ultrasound probe 3. In such embodiment, the probe cap 13 can be used as an exchangeable hygienic barrier between the housing 12 of the ultrasound probe 3 and the human or animal body. In this embodiment, the ultrasound signal should be transmitted through the material of the probe cap 13. The probe cap 13 may for example be made of plastic material, such as polypropylene which has a high transmission of ultrasound waves. It is also possible to provide an additional means or device in the probe cap 13, such as a gel pad, at the location aligned with the sensor measurement area, to improve the transmission of the ultrasound waves through the probe cap 13.

FIG. 3 shows a second embodiment of an ultrasound probe 3 according to the invention. On the housing 12 a probe cap 13 is provided. The probe cap 13 is for example connected to the housing 12 by a click-fit connection. The ultrasound probe 3 comprises a housing 12 having a sensor measurement area 18 which is completely exposed by an opening 19 provided in the probe cape 13. The probe cap 13 comprises a first protrusion 14 and a second protrusion 16. The first protrusion 14 and the second protrusion 16 are arranged at a line extending parallel to the sensor measurement area direction, wherein the line is spaced from the sensor measurement area 18. In this embodiment, the sensor measurement area 18 is not obstructed by the first protrusion 14 and the second protrusion 16.

This position of the first protrusion 14 and the second protrusion 16 has the further advantage that by rotation of the ultrasound probe 3 around the sensor measurement area direction, a pressure difference between a first pressure with which the sensor measurement area and a second pressure with which the first protrusion 14 and the second protrusion 16 are pressed against the skin S of the human or animal body can be adapted. This first pressure is related to the measurement quality of the ultrasound probe 3, while the second pressure is related to the degree of stabilization of the blood vessel V but also the degree of compression of the blood vessel V. By rotation of the ultrasound probe 3 around the sensor measurement area direction this pressure difference can be controlled to optimize the first pressure and the second pressure for a specific application.

FIG. 4 shows another example of an ultrasound probe 3 having the first protrusion 14 and the second protrusion 16 arranged at a line parallel to but spaced from the sensor measurement area 18. Rotation in direction R1 of the ultrasound probe 3 around the sensor measurement area direction will relatively increase the pressure with which the first and second protrusions 14, 16 are pressed on the skin S, while rotation in direction R2 will relative increase the pressure with which the sensor measurement area 18 is pressed on the skin S of the human or animal body.

FIG. 5 shows a third embodiment of an ultrasound probe 3 according to the invention. The ultrasound probe 3 of this embodiment does not comprise an exchangeable probe cap. The first protrusion 14 and the second protrusion 16 are an integral part of the housing 12. The first protrusion 14 and the second protrusion 16 define a recess 17 therebetween. The sensor measurement area 18 is arranged in the base of the recess 17. When the first protrusion 14 and the second protrusion 16 are pressed on the skin S at opposite sides of the blood vessel V, the blood vessel V will be arranged in a stabilized position between the first protrusion 14 and the second protrusion 16. In an alternative embodiment only one protrusion may be provided. The ultrasound probe 3 may be positioned such that the blood vessel V is pushed against this one protrusion to stabilize the blood vessel V, when the ultrasound probe 3, in particular its contact surface 3b is pressed on the skin S of the human or animal body.

The cannula insertion system 1 may comprise one or more force sensors 21, as shown in FIG. 1, that are arranged to measure the force with which the first protrusion 14 and/or the second protrusion 16 are pressed on the skin S of the human or animal body. On the basis of this measured force(s) and/or the ultrasound image signal obtained by the ultrasound probe the shape of the cross section of the blood vessel V may be monitored. In particular, it can be determined whether the force is not too low such that the there is an increased risk on rolling of a blood vessel and/or whether the force is not too high such that there is a risk that the blood vessel is compressed too much for insertion of a cannula into the blood vessel. The one or more force sensors 21 may be provided in the ultrasound probe 3 as shown in FIG. 1 or at any other suitable location where the force with which the first protrusion 14 and/or the second protrusion 16 are pressed on the skin S of the human or animal body can be determined.

The cannula insertion system 1 may be used as follows.

A suitable blood vessel V and an associated insertion location for insertion of the cannula 2 are determined. The cannula insertion system 1 is controlled to insert the cannula 2 in the blood vessel V. During insertion of the cannula 2 into the blood vessel V the position of the blood vessel V is monitored in the ultrasound image constructed on the basis of the ultrasound signal provided by the ultrasound probe 3. When it is determined that the position of the blood vessel changes, for example that movement of the blood vessel in a lateral direction with respect to the skin exceeds a certain distance, it may be determined that the blood vessel V has rolled sideways. In such case, insertion of the cannula 2 is stopped to prevent incorrect insertion of the cannula 2. The cannula 2 may be retracted, for example to a location proximate to the skin (just above or just below the skin) or above the skin and spaced from the wall of the blood vessel V.

Then, insertion of the cannula 2 into the blood vessel V may be retried.

In dependence of the direction of the lateral movement of the blood vessel V the position and/or orientation of the ultrasound probe 3 may be adapted to increase the chance of a successful insertion of the cannula 2 into the blood vessel V. Advantageously, the longitudinal axis of the cannula 2, and therewith the insertion direction ID is arranged in a plane extending in the longitudinal direction and the radial direction of the blood vessel V near the intended insertion location of the cannula 2 into the blood vessel V.

In addition, or alternatively, the ultrasound probe 3 may be controlled to increase the pressure with which the ultrasound probe 3 is pressed on the skin and/or the ultrasound probe 3 may be rotated about the longitudinal axis of the blood vessel V to improve the stabilization of the blood vessel V by the ultrasound probe 3. Then the cannula 2 may again be moved towards the blood vessel V to insert the cannula 2 into the blood vessel V.

These steps may be repeated until the cannula 2 is successfully inserted into the blood vessel V. If insertion is not successful after a predetermined number of insertion attempts, the cannula insertion procedure may be aborted.

It is also possible to adapt the insertion direction ID of the cannula 2 itself to improve the chance on a successful insertion of the cannula 2. The cannula insertion direction ID may be adapted by translation and/or rotation of the end-effector 4 that supports both the cannula 2 and the ultrasound probe 3, or by translation and/or rotation of only the cannula insertion device 8, or part thereof, independently of the position/orientation of the ultrasound probe 3.

The cannula insertion device 8 may for example have an additional positioning system or the cannula insertion device 8 and the ultrasound probe 3 may each have their own positioning system to allow positioning of the insertion direction ID of the cannula insertion device 8 with respect to the ultrasound probe 3. It is also possible that the cannula insertion device 8 and the ultrasound probe 3 have a common positioning system for combined movements, and one or more further positioning systems mounted on the common positioning system for separate movement of the cannula 2 or the ultrasound probe 3, respectively. For instance, a further positioning system may be provided for adjusting the insertion direction ID of the cannula 2 without the need to adapt the position of the ultrasound probe 3.

The insertion direction ID may be, dependent on the location of the blood vessel V to be inserted, be adapted in one or more rotation angles. This will for example allow to adapt the angle with which the insertion direction ID is arranged with respect to the surface of the skin S or the angle with which the insertion direction ID is arranged with respect to an imaging plane of the ultrasound probe 3.

In these embodiments, the cannula 2 may be retracted to a location proximate to the skin or above the skin, before the insertion direction ID is adapted. Translation and/or rotation of the end-effector 4 and/or at least a part of the cannula insertion device 8 will adapt the orientation of the insertion direction. The insertion direction ID may for example be adjusted to a new position of the blood vessel V after a lateral movement of the blood vessel V. For instance, the insertion direction ID may be adjusted to extend more closely to a plane extending in a longitudinal direction and a radial direction of the rolled blood vessel V. The actual position of the blood vessel V may be determined by using the ultrasound images obtained by the ultrasound probe 3. In this respect, it may be advantageous to adapt the insertion direction ID by movement of the cannula insertion device 8, or a part thereof, and thus without movement of the ultrasound probe 3. This enables the ultrasound probe 3 to continuously monitor the actual position of the blood vessel V and at the same time stabilize the blood vessel V with the pressure with which the ultrasound probe 3 is pressed on the human or animal body using the one or more protrusions of the ultrasound probe 3.

1. A cannula insertion system to insert a cannula into a blood vessel of a human or animal body, comprising:
an ultrasound probe to provide an ultrasound signal to determine a location of a blood vessel in a human or animal body; and
a cannula insertion device configured to insert the cannula into the blood vessel;
wherein the ultrasound probe comprises a contact surface to contact a skin of the human or animal body, and
wherein the contact surface comprises one or more protrusions that can be used to stabilize the blood vessel when the contact surface is pressed on the skin.

2. The cannula insertion system of claim 1, wherein the one or more protrusions comprise a first protrusion and a second protrusion, wherein the first protrusion and the second protrusion are spaced with respect to each other to provide a recess therebetween in which a blood vessel can be stabilized.

3. The cannula insertion system of claim 2, wherein the ultrasound probe comprises an elongate sensor measurement area extending in a sensor measurement area direction, wherein the first protrusion and the second protrusion are arranged on a line extending parallel to the sensor measurement area direction.

4. The cannula insertion system of claim 3, wherein the first protrusion and the second protrusion are arranged on the sensor measurement area.

5. The cannula insertion system of claim 3, wherein the line extending parallel to the sensor measurement area direction is spaced from the sensor measurement area.

6. The cannula insertion system of claim 2, wherein the cannula insertion device is arranged to insert the cannula into the blood vessel at a cannula insertion location, and wherein the first protrusion and the second protrusion are positioned to be pressed on the skin at opposite sides of the cannula insertion location.

7. The cannula insertion system of claim 2, wherein the cannula insertion system further comprises:
a cannula insertion device positioning system to support and position the cannula insertion device;
an ultrasound probe positioning system to support and position the ultrasound probe; and
a control device arranged to control the cannula insertion device positioning system and the ultrasound probe positioning system such that during insertion of the cannula into the blood vessel, the cannula insertion device positioning system positions the cannula insertion device to insert the cannula into the blood vessel at a cannula insertion location and the ultrasound probe positioning system positions the first protrusion and the second protrusion on the skin at opposite sides of the cannula insertion location.

8. The cannula insertion system of claim 1, wherein the ultrasound probe comprises a housing and an exchangeable probe cap mounted on the housing and forming at least part of the contact surface of the ultrasound probe.

9. The cannula insertion system of claim 8, wherein the one or more protrusions are integrated in the probe cap.

10. The cannula insertion system of claim 8, wherein the one or more protrusions are integrated in the housing and the probe cap covers the one or more protrusions.

11. The cannula insertion system of claim 8, wherein the probe cap completely forms the contact surface of the ultrasound probe.

12. The cannula insertion system of claim 8, wherein the probe cap comprises an opening to partially or completely expose a sensor measurement area of the ultrasound probe.

13. The cannula insertion system of claim 8, wherein the probe cap is an exchangeable hygienic barrier between the housing of the ultrasound probe and the human or animal body.

14. The cannula insertion system of claim 1, wherein the ultrasound probe comprises a housing and wherein the one or more protrusions are integrated in the housing.

15. The cannula insertion system of claim 1, wherein the cannula insertion system comprises one or more force sensors that are arranged to measure a force with which the one or more protrusions are pressed on the skin of the human or animal body.

16. The cannula insertion system of claim 1, wherein the cannula insertion system is arranged to control a pressure with which the ultrasound probe is pressed on the human or animal body on the basis of a value representative for a non-roundness of the blood vessel.

17. An ultrasound probe to provide an ultrasound signal to determine a location of a blood vessel in a human or animal body, wherein the ultrasound probe comprises a contact surface to contact a skin of the human or animal body, wherein the contact surface comprises one or more protrusions that can be used to stabilize the blood vessel when the contact surface is pressed on the skin.
